(19) United States
(12) Patent Application Publication
(10) Pub. No.: US 2024/0000044 A1
(43) Pub. Date: Jan. 4, 2024

(54) SYSTEM FOR MONITORING PETS

(71) Applicants: MAX.K KOREA Inc., Seoul (KR); Max K LLC, Houston, TX (US)

(72) Inventors: Kibun Max KOO, Houston, TX (US); Sang Min AN, Seoul (KR)

(73) Assignees: MAX.K KOREA Inc., Seoul (KR); Max K LLC, Houston, TX (US)

(21) Appl. No.: 18/181,921

(22) Filed: Mar. 10, 2023

(30) Foreign Application Priority Data
Jun. 30, 2022 (KR) 10-2022-0080142

(51) Int. Cl. A01K 29/00; H04W 4/30; A01K 11/00

(52) U.S. Cl. CPC A01K 29/005; H04W 4/30; A01K 11/008

(57) ABSTRACT

The present disclosure relates to systems and methods for monitoring pets. More particularly, the system being configured such that, in order to remotely monitor behavior or condition of a pet, a wearable device is worn around a body portion of the pet, a signal detected by the wearable device is registered to a pet monitoring server via a repeater, and a user can access the pet monitoring server with a user terminal to remotely monitor the behavior or condition of the pet.

### CROSS REFERENCE TO RELATED APPLICATION

This application claims priority to Korea Patent Application No. 10-2022-0080142 filed on Jun. 30, 2022, the content of which is expressly incorporated by reference in its entirety.

### TECHNICAL FIELD

The present disclosure relates to a system for monitoring pets. More particularly, the system is configured such that, in order to remotely monitor behavior or condition of a pet, a wearable device is worn around a body portion of a pet, a signal detected by the wearable device is registered to a pet monitoring server via a repeater, and a user can access the pet monitoring server with a user terminal to remotely monitor the behavior or condition of the pet.

### BACKGROUND

There is a social phenomenon in which various types of companion animals (hereinafter, which is referred to as pets), including dogs and cats, share a living environment with people. According to the social phenomenon, people and pets naturally share a living space with each other and lead a life together. In addition, due to aging, a smaller type family, and an increase in single-person households, more and more households consider pets as family members. Careful caring of a pet is required, but a method of the most people of caring for a pet is very simple. For example, the pet caring method is limited to providing a simple caring function such as automatic feeding every predetermined time for a pet left alone when a companion person (which is referred to as an owner) goes out or providing an image of the owner to the pet.

However, in the case of simply providing food or providing repeated images, the pet may initially respond, but after learning over time, the pet does not respond to the caring method, so the user cannot obtain the effect of caring for the pet.

Furthermore, there is no way for the user to check the health status of the pet with the existing caring method using food discharge or video provision. In a situation where the user cannot be with the pet due to work or going out, the pet may show abnormal behavior due to anxiety and stress, so it is necessary to check the behavior or condition of the pet left alone.

For example, the most representative monitoring method currently used is to equip a camera at a predetermined location in the house, and to allow the owner to connect to the camera with a user terminal of the owner to check the health condition of the pet from a remote location at any time, and according to a result of the check, to take immediate action when the pet is in an abnormal state.

However, in such a monitoring method, the devices such as the camera provided in the house and the user terminal must be directly connected to each other in real time. Therefore, there is a problem that expensive communication costs are required for pet monitoring and the communication can be affected by traffic during busy time periods.

Korean Patent No. 2139922 (Jul. 31, 2020) relates to a behavior monitoring care system for a companion animal, and a wearable device and an application of a mobile terminal interwork with each other to generate an avatar emoticon in the form of a character and to express behavior of a companion animal as an avatar emoticon in the form of a character.

Korean Patent Application Publication No. 2022-0055518 (May 5, 2022) relates to a system and a method for monitoring a companion animal based on companion animal status information, and a companion animal tracking device attached to a companion animal to obtain motion information about the companion animal.

There is a need and a desire for improved systems and methods for pet monitoring.

### BRIEF SUMMARY OF THE EMBODIMENTS

According to an embodiment of the present disclosure, a system for monitoring pets includes: a pet monitoring server, wherein the pet monitoring server includes: a first communication access part configured to perform communication access to a wearable device worn around a pet; a second communication access part configured to perform communication access to a user terminal, wherein the pet monitoring server is configured to: receive detection information including biometric information, movement information, environmental information, or combination thereof of the pet, detected by the wearable device; classify and register the received detection information by each pet; and provide the registered detection information of the pet according to a request of the user terminal, and wherein the first communication access part and the second communication access part perform communication access through different communication sessions.

The wearable device is configured to perform communication access to the first communication access part via a repeater including a base station or an access point, and wherein the first communication access part includes a low power wide area network including at least one of LoRa (long range), LTE cat (long term evolution category), Wi-Fi, or NB-IOT (narrow band internet of things).

The user terminal is configured to perform communication access to the second communication access part in a web-based or push/pull manner, and wherein the second communication assess unit is configured to include wide area wired or wireless network including at least one of 5G, Wi-Fi, wired or wireless Ethernet, CDMA, or LTE.

The pet monitoring server is further configured to receive the detection information from the wearable device via a non-subscribed user terminal not subscribed in a paid communication service as a repeater, and wherein the non-subscribed user terminal is configured to receive the detection information from the wearable device via near-field communication (NFC), BLE, or Bluetooth, and perform communication access only via Wi-Fi in a process of transmitting the information to the first communication access part.

The pet monitoring server is further configured to receive the detection information from the wearable device via a subscribed user terminal subscribed in the paid communication service as a repeater, and wherein the subscribed user terminal is configured to receive the detection information from the wearable device in background environment via near-field communication (NFC), BLE, or Bluetooth, and perform communication access via 5G, Wi-Fi, wired and wireless Ethernet, CDMA, or LTE, in a process of transmitting the information to the first communication access part.

The detection information includes biometric information, movement information, or environmental information, which is detected by the non-wearable device and is transmitted from the non-wearable device to the wearable device via near-field communication (NFC), BLE, or Bluetooth, and wherein the non-wearable device includes: a pet house, dishes, toys, health measuring devices, or medical devices.

According to embodiments, the pet monitoring server is further configured to receive the detection information from the wearable device of each pet with respect to a plurality of pets located in the same space, and wherein the detection information transmitted from the non-wearable device to the wearable device of a specific pet is configured to be classified into the detection information of the specific pet by the specific wearable device and be stored.

In addition, according to an embodiment of the present disclosure, a method for monitoring pets including: receiving, in a system for monitoring pets, detection information including biometric information, movement information, environmental information, or combination thereof of a pet, detected by a wearable device via a first communication access part; registering the received detection information by each pet; and transmitting the registered detection information according to a request of a user terminal performing communication access via a second communication access part, wherein the first communication access part and the second communication access part are configured to perform communication access through different communication sessions.

The method further includes performing communication access with the first communication access part via a repeater including a base station or an access point; and wherein the first communication access part includes a low power wide area network including at least one of LoRa (long range), LTE cat (long term evolution category), Wi-Fi, and NB-IOT (narrow band internet of things).

The method further includes the user terminal configured to perform communication access to the second communication access part in a web-based or push/pull manner, and wherein the second communication assess unit is configured to include wide area wired or wireless network including at least one of 5G, Wi-Fi, wired or wireless Ethernet, CDMA, and LTE.

The method further includes receiving, in the system for monitoring pets, the detection information from the wearable device via a non-subscribed user terminal not subscribed in a paid communication service as a repeater, wherein the non-subscribed user terminal is configured to receive the detection information from the wearable device via near-field communication (NFC), BLE, or Bluetooth, and wherein the non-subscribed user terminal is configured to perform communication access only via Wi-Fi, in a process of transmitting the information to the first communication access part.

The method further includes receiving, in the system for monitoring pets, the detection information from the wearable device via a subscribed user terminal subscribed in a paid communication service as a repeater, and wherein the subscribed user terminal is configured to perform communication access via 5G, Wi-Fi, wired and wireless Ethernet, CDMA, or LTE, in a process of receiving the detection information in background environment from the wearable device via near-field communication (NFC), BLE, or Bluetooth, and transmitting the information to the first communication access part.

The detection information further includes biometric information, movement information, or environmental information, which is detected by the non-wearable device and is transmitted from the non-wearable device to the wearable device via near-field communication (NFC), BLE, or Bluetooth, and wherein the non-wearable device includes: a pet house, dishes, toys, health measuring devices, or medical devices.

The method further includes separately receiving, in the system for monitoring pets, the detection information from the wearable device of each pet, with respect to a plurality of pets located in the same space, and wherein the detection information transmitted from the non-wearable device to the wearable device of the pet is configured to be classified into the detection information of the pet by the wearable device and be stored.

As described above, according to the system for monitoring pets of the present disclosure, a signal detected by the wearable device worn around the body portion of the pet is registered to the pet monitoring server via the repeater. The user accesses the pet monitoring server with the user terminal to remotely monitor the behavior or condition of the pet. Therefore, the user can monitor the behavior or condition of the pet at minimal costs.

Furthermore, according to the present disclosure, when a pet uses the non-wearable device capable of being used in common, the wearable device worn around the pet receives information transmitted from the non-wearable device via near-field communication (NFC), Bluetooth, BLE, etc., and registers the information into the pet monitoring server along with the biometric information, movement information, environmental information, etc. detected by itself. Accordingly, the user can access the pet monitoring server to perform extensive and diverse monitoring of the behavior and condition of the pet.

Furthermore, according to the present disclosure, it is possible to prevent occurrence of interdependence between the wearable device worn around the pet and the user terminal, to prevent influence on communication traffic, and to reduce the communication cost, thereby minimizing restrictions on time and place. Accordingly, it is possible to freely monitor the behavior or condition of the pet as much as possible.

Other aspects, features, and techniques will be apparent to one skilled in the relevant art in view of the following detailed description of the embodiments.

### BRIEF DESCRIPTION OF THE DRAWINGS

The features, objects, and advantages of the present disclosure will become more apparent from the detailed description set forth below when taken in conjunction with the drawings in which like reference characters identify correspondingly throughout and wherein:

FIG. 1 is a view showing the entire structure of a system for monitoring pets according to an embodiment of the present disclosure;

FIG. 2 is a block diagram showing a structure of a pet monitoring server according to the embodiment of the present disclosure in detail;

FIG. 3 is a view showing a structure of a wearable device according to the embodiment of the present disclosure in detail;

FIG. 4 is a view showing a process of transmitting, by the wearable device according to the embodiment of the present disclosure, information detected by a non-wearable device and information directly directed from a pet to a pet monitoring server; and

FIG. 5 is a flowchart showing an operation process of a pet monitoring method according to an embodiment of the present disclosure.

### DETAILED DESCRIPTION OF THE EXEMPLARY EMBODIMENTS

### Overview and Terminology

Hereinbelow, an exemplary embodiment of a system for monitoring pets according to the present disclosure will be described with reference to accompanying drawings. Throughout the drawings, the same reference numerals will refer to the same or like elements or parts. Furthermore, the structural or functional description specified to exemplary embodiments of the present disclosure is intended to describe the exemplary embodiment according to the present disclosure. In the flowing description, unless otherwise defined, all terms including technical and scientific terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The present disclosure proposes a method in which the behavior or condition of the pet can be monitored freely with minimum costs by registering biometric information or movement information detected by the wearable device worn around the body portion of the pet to the pet monitoring server via a repeater, and accessing the pet monitoring server with the user terminal to check the registered information. The method may address conventional systems problems of a user checking the behavior or condition of the pet in a non-real time manner.

Specifically, the present disclosure is configured such that, when among a plurality of pets located in a predetermined space, any one specific pet uses a non-wearable device including other common devices (e.g., toys, pet medical devices, etc.) besides a wearable device of the specific pet, the wearable device worn around the specific pet receives information transmitted from the corresponding non-wearable device via near-field communication including RFID, Bluetooth, Bluetooth low energy (BLE), etc. and registers the information to a pet monitoring server along with biometric information, movement information, environmental information (sound, peripheral temperature, etc.) detected by the wearable device, thereby allowing the user to access the pet monitoring server and widely and diversely monitor the behavior or condition of the pet.

The present disclosure is proposed to solve the above problems, and the present disclosure is intended to provide a wearable device to a body portion of a pet, to register biometric information, movement information, environmental information, etc., detected by the wearable device, to a pet monitoring server via a repeater, and to access, by a user with a user terminal, the pet monitoring server to remotely monitor behavior or condition of the pet. In other words, the present disclosure is configured to allow the wearable device worn around the pet and the user terminal to use separate networks, and to register the information detected from the pet to the pet monitoring server, and to monitor behavior or condition of the pet by checking the pet information registered in the pet monitoring server.

Furthermore, another objective of the present disclosure is to allow the wearable device worn around a pet to register biometric information, movement information, environmental information, etc. of a pet to the pet monitoring server, by using low power wide area (LPWA) networks such as LoRa (long range), LTE cat (long term evolution category evolution category), NB-IoT (narrow band internet of things), etc., with low communication costs.

Furthermore, a further objective of the present disclosure is to use a user terminal non-subscribed in a paid communication service to register biometric information, movement information, environmental information, etc. of a pet, which are detected by the wearable device worn around the pet, to the pet monitoring server via Wi-Fi.

Furthermore, a further objective of the present disclosure is to register biometric information, movement information, environmental information, etc. of a pet, which are detected by the wearable device worn around the pet, to the pet monitoring server via a user terminal subscribed in the paid communication service as a repeater.

Furthermore, a further objective of the present disclosure is to allow, in a situation where a user cares for a plurality of pets or a plurality of pets exists in the same space, when any one pet uses a non-wearable device used in common, the wearable device worn around the specific pet to receive usage information transmitted from the non-wearable device via near-field communication (NFC), Bluetooth, BLE, etc., and to register the information to the pet monitoring server along with the biometric information, movement information, environmental information, etc. of the corresponding pet.

Furthermore, a further objective of the present disclosure is to prevent the wearable device worn around a pet and a user terminal from being interdependent, from being affected on communication traffic, to reduce the communication cost, and minimize restrictions on time and place, thereby freely monitoring behavior or condition of the pet as much as possible.

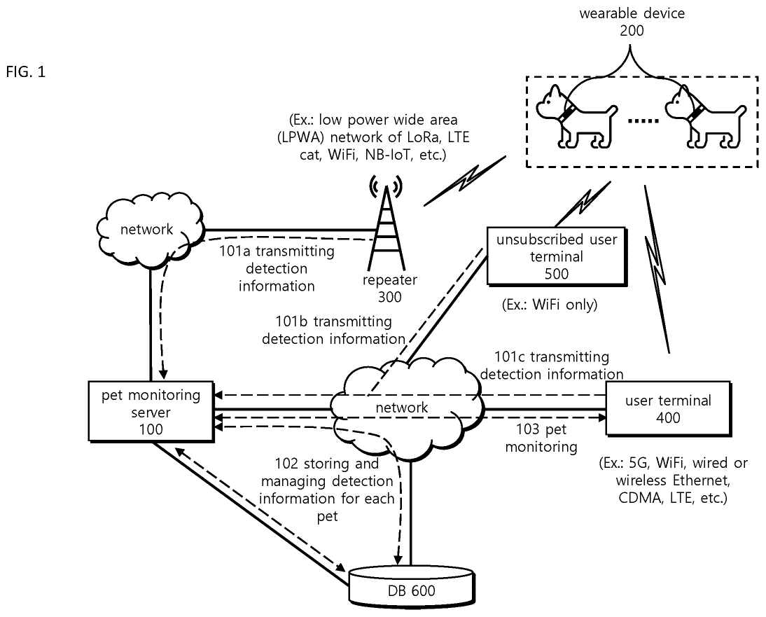

FIG. 1 is a view showing the entire structure of a system for monitoring pets according to an embodiment of the present disclosure.

As shown in FIG. 1, the system for monitoring pets according to the embodiment of the present disclosure includes a pet monitoring server 100, a wearable device 200, a repeater 300, a user terminal 400, a non-subscribed user terminal 500, and a database 600.

The pet monitoring server 100 is configured to receive, from the wearable device 200 worn around a body portion of a pet, detection information including variety of biometric information such as body temperature, pulse, etc. directly measured from the pet, movement information of the pet, environmental information such as sound and periphery temperature, or combination thereof via a network, 101a. Subsequently, the detection information received from the wearable device 200 is stored and managed in the database 600 while being classified by each pet, 102.

Here, the pet monitoring server 100 receives the detection information including the biometric information, the movement information, and the environmental information of the pet from the wearable device 200 via the repeater 300. The repeater 300 includes a base station or an access point connected to low power wide area (LPWA) networks, including LoRa, LTE cat, NB-IoT, etc. with low communication costs.

Furthermore, when the pet monitoring server 100 receives the detection information of the pet from the wearable device 200 via the repeater 300, the pet monitoring server 100 can obtain an image signal capturing movement of the pet inside the house and a sound signal including barking via an independent device with a camera or a microphone inside the house. The pet monitoring server 100 receives the obtained image or sound signal through the wearable device 200 and the repeater 300.

Furthermore, in the process in which the detection information for each pet is stored and managed in the database 600, the pet monitoring server 100 provides the detection information of the pet to the user terminal 400 according to a request of the user terminal 400 performing communication access via the network to allow behavior or condition of the pet to be monitored, 103.

Here, the pet monitoring server 100 may proceed to achieve communication access to the user terminal 400 via other networks different from the wearable device 200, which are wide area networks such as 5G, Wi-Fi, wired or wireless Ethernet, CDMA, and LTE. The pet monitoring server 100 may provide the detection information for pet monitoring, the information being stored and managed in the database 600, to the user terminal 400.

As described above, in the process of communication access and receiving and transmitting of the detection information between the pet monitoring server 100 and the wearable device 200 or the user terminal 400, usage of the separate different networks (or performing communication access via different communication sessions) is one of characteristics of the present disclosure. Focusing on a fact that an emergency does not occur even if a user who cares for a plurality of pets such as dogs or cats does not check the condition of the pet in non-real time, and when the pet monitoring server 100 receives and registers the detection information by each pet, there is no need to use the wide area networks with high communication costs, and the LPWA networks available at low communication costs can be used.

Accordingly, the wearable device 200 and the user terminal 400 are not greatly affected by communication traffic, and communication costs of pet monitoring can be reduced. Furthermore, the wearable device 200 does not transmit the detection information about the behavior or condition of the pet to the user terminal 400 owned by the user, the owner of the pet, in real time, but transmits and registers the information to the pet monitoring server 100 via the repeater 300. The user can check the pre-registered detection information by web accessing the pet monitoring server 100 at a convenient time and place, so that the user can freely monitor the behavior or condition of the pet.

Furthermore, the pet monitoring server 100 may adopt various methods as follows, in addition to the above-described method of receiving the detection information of the pet from the wearable device 200 via the repeater 300 using the LPWA networks.

For example, when the user has the non-subscribed user terminal 500 that is not subscribed in a paid communication service in the house, the pet monitoring server 100 may receive and register the detection information of the pet detected by the wearable device 200 via the non-subscribed user terminal 500, 101b.

In other words, the non-subscribed user terminal 500 is a smart phone using only Wi-Fi, and when the detection information of the pet is transmitted from the wearable device 200 while a pre-set application program is running, the detection information may be transmitted to the pet monitoring server 100 via Wi-Fi. When the non-subscribed user terminal 500 is used as described above, it is an advantage that no extra cost is required.

Furthermore, when the user terminal 400 owned by the user is located near the wearable device 200, the pet monitoring server 100 may receive and register the detection information of the pet detected by the wearable device 200 via the user terminal 400, 101c. Here, the user terminal 400 is a subscribed user terminal subscribed in a paid communication service, and not only the user terminal owned by the owner of the pet but also user terminals owned by other family members can be used.

In other words, in the user terminal 400 located at periphery of the wearable device 200, while the hidden-type application program is running, the detection information of the pet transmitted from the wearable device 200 is transmitted to the pet monitoring server 100 in the communication method (5G, LTE, Wi-Fi, etc.) used in the user terminal 400. In this process, the user terminal 400 may be preset not to directly check the detection information of the pet transmitted from the wearable device 200.

Here, via the subscribed or non-subscribed user terminal as a repeater, the wearable device 200 of the pet perform communication access to the pet monitoring server. Furthermore, even when the communication access for the subscribed user terminal to receive the detection information from the pet monitoring server passes through the same type of networks, the communication accesses are configured to be performed through different sessions.

The wearable device 200 is configured to be fastened to and used at a body portion such as the neck, leg, torso of the pet, and includes various sensors including a biometric detection sensor, a movement detection sensor, a temperature detection sensor, a sound detection sensor, a camera sensor, etc. and a GPS receiver for positioning.

Furthermore, the wearable device 200 collects the detection information including not only the biometric information about the temperature and pulse of the pet, but also the movement information according to movement and behavior in the indoor and outdoor space, and the environmental information about periphery temperature or sound generated by the pet, periodically. The wearable device 200 transmits the collected detection information to the pet monitoring server 100 via the repeater 300, thereby registering the behavior or condition of the pet.

Meanwhile, in a situation where the user cares a plurality of pets instead of one in the house, or a plurality of pets exists in the same space, when any one of non-wearable devices including common devices such as pet toys and pet medical devices that is usable by any one specific pet in common is used, there is a need to provide and register the information according to usage of the non-wearable device is provided to the pet monitoring server 100.

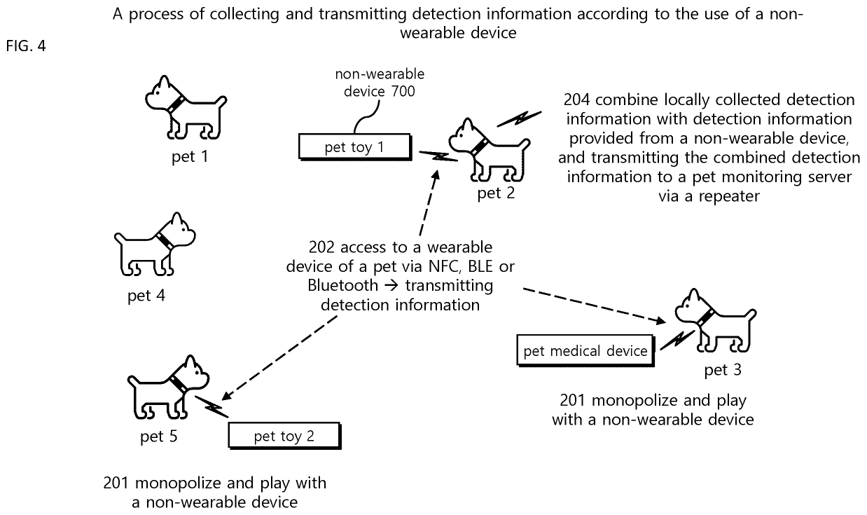

In other words, the wearable device 200 receives the usage information generated as the pet uses the non-wearable device (e.g., moving distance for toys, captured images or inspection data for medical devices, etc.) from the non-wearable device via near-field communication (NFC), Bluetooth, BLE, etc. The wearable device 200 transmits the received usage information of the non-wearable device along with the detection information directly measured from the pet to the pet monitoring server 100 via the repeater 300, so that the information can be registered (referring to FIG. 4).

The above-described operation is one of other characteristics of the present disclosure, in a situation in which a plurality of non-wearable devices used by a plurality of pets in common exists, when each pet uses any one non-wearable device, the wearable device 200 worn around each pet receives information about how the pet used which non-wearable device from the non-wearable device, and provides the information to the pet monitoring server 100 and resisters the information. Accordingly, a region in which the behavior and condition of the pet can be monitored expands to various toys, medical devices, etc. by simultaneously utilizing the detection information collected by itself and the information collected through the non-wearable device, so that extensive and close monitoring can be performed.

The user terminal 400 is a communication terminal such as a smartphone or tablet owned by the user who is a pet owner of the pet, and the user terminal 400 performs communication access to the pet monitoring server 100 through web-based manner or push/pull manner, and receives the detection information stored in the database 600 from the pet monitoring server 100 to monitor the behavior or condition of the pet.

At this time, the detection information provided from the pet monitoring server 100 is registered information provided from the wearable device 200 before present time and accumulated over time. In addition, the user located in a remote place can check behavior amount and physical condition of the pet located in the house or other places with the detection information.

Meanwhile, although not shown in FIG. 1, when the LPWA networks repeater is configured in combination with a server computer function, the wearable device worn around each pet located within a communication range (e.g., 3 Km to 10 Km) of the repeater and the user terminal are connected to each other through the repeater without a separate communication cost, thereby performing the monitoring with respect to the behavior and condition of the pet.

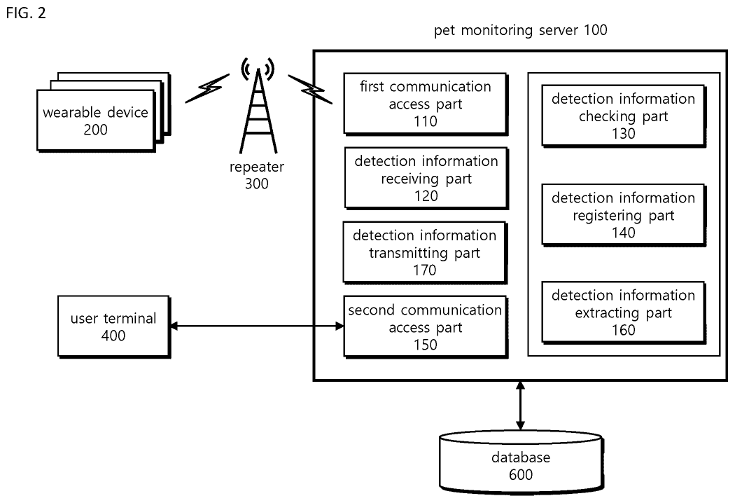

FIG. 2 is a block diagram showing a structure of a pet monitoring server according to the embodiment of the present disclosure in detail.

As shown in FIG. 2, the pet monitoring server 100 includes a first communication access part 110, a detection information receiving part 120, a detection information checking part 130, a detection information registering part 140, a second communication access part 150, a detection information extracting part 160, a detection information transmitting part 170, and the like.

The first communication access part 110 performs communication access to the wearable device 200 via the repeater 300, which includes a base station or an access point. At this point, the first communication access part 110 performs communication access with the wearable device 200 via the LPWA networks such as LoRa, LTE cat, Wi-Fi, NB-IoT, and the like.

The detection information receiving part 120 receives the detection information including the biometric information, the movement information, the environmental information of the pet, or the combination thereof from the wearable device 200 performing communication access via the first communication access part 110.

Here, the detection information received from the wearable device 200 to the detection information receiving part 120 may include the detection information such as the biometric information, the movement information, the environmental information, etc., which is detected by the non-wearable device and is transmitted from the non-wearable device to the wearable device 200 via near-field communication (NFC), Bluetooth, BLE, and the like.

For example, the wearable device 200 receives the detection information about the behavior and condition of the pet of the pet using the non-wearable device including a house, dishes, toys, health measuring devices, medical devices, etc. of the pet. from the non-wearable device. The received detection information detected by the non-wearable device may be transmitted to the pet monitoring server 100 along with the detection information directly obtained from the pet.

Furthermore, from the wearable device 200 with respect to a plurality of pets existing in the same space, the detection information receiving part 120 may receive the detection information corresponding to the pet, separately. At this point, the detection information transmitted from the non-wearable device to the wearable device 200 of a specific pet may be classified into detection information of the specific pet by the wearable device and stored.

The detection information checking part 130 distinguishes whether the detection information transmitted from the detection information receiving part 120 is detected from which pet, and inputs a result thereof into the detection information registering part 140.

At this point, the detection information transmitted from the detection information receiving part 120 includes an identification number uniquely assigned to each wearable device 200. Therefore, the detection information checking part 130 can check whether the detection information is transmitted from the wearable device 200 of which pet, on the basis of the identification number.

Here, the detection information has been analyzed on the basis of the measured value obtained by the wearable device 200 from the pet, but may be a raw data as measured values prior to performing analysis. In this case, the detection information checking part 130 analyzes the raw data about the biometric information, the movement information, the environmental information, etc. transmitted from the wearable device 200, and then may provide an analyzed result (i.e., detection information of corresponding pet) to the detection information registering part 140.

The detection information registering part 140 performs registration of the detection information separated for each pet, which is checked by the detection information checking part 130. In other words, the detection information registering part 140 allows the detection information for each pet to be stored and managed in the database 600.

The second communication access part 150 performs communication access to the user terminal 400 owned by the user (including other family members caring for the pet), who is an owner of the pet, through the web-based or push/pull manner.

In other words, the second communication access part 150 performs communication access to the user terminal 400 via networks different from the wearable device 200 performing communication access to the first communication access part 110 via the LPWA networks, i.e., the wide area networks including 5G, Wi-Fi, wired or wireless Ethernet, CDMA, and LTE. Here, a concept of the communication access performed through different networks includes difference of (network) sessions. In other words, even though the first communication access part and the second communication access part access to the same type of networks, the communication access is performed with sessions thereof different from each other.

When the detection information for monitoring a pet is requested by the user terminal 400 performing communication access via the second communication access part 150, the detection information extracting part 160 extracts the detection information of the corresponding pet, which is stored and managed in the database 600, and transmits the extracted detection information to the detection information transmitting part 170.

According to a request of the user terminal 400, the detection information transmitting part 170 transmits the detection information of the pet extracted by the detection information extracting part 160 to the user terminal 400 via the second communication access part 150.

Meanwhile, the pet monitoring server 100 may receive the detection information of the pet from the wearable device 200 via the non-subscribed user terminal 500 (i.e., terminal that is not subscribed to a paid communication service) serving as a repeater. In this process, the non-subscribed user terminal 500 receives the detection information from the wearable device 200 via near-field communication (NFC), Bluetooth, BLE, etc. The non-subscribed user terminal 500 may perform communication access to the first communication access part 110 only via Wi-Fi in a process of transmitting the received detection information to the pet monitoring server 100.

Furthermore, the pet monitoring server 100 may receive the detection information of the corresponding pet from the wearable device 200 via the user terminal 400 serving as a repeater. In this process, the user terminal 400 in the background environment (i.e., a state in which a hidden-state application program is running) receives the detection information from the wearable device 200 via near-field communication (NFC), Bluetooth, BLE, etc. In addition, in a process in which the received detection information is transmitted to the pet monitoring server 100, the user terminal 400 may perform communication access to the first communication access part 110 via the wide area networks such as 5G, Wi-Fi, wired or wireless Ethernet, CDMA, LTE, etc.

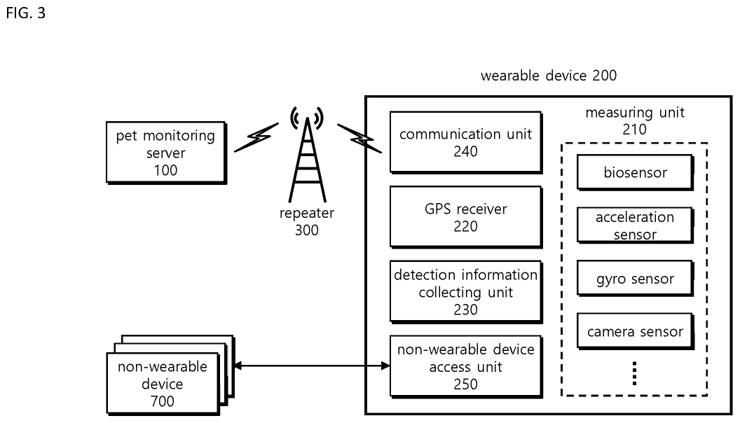

FIG. 3 is a view showing a structure of the wearable device according to the embodiment of the present disclosure in detail.

As shown in FIG. 3, the wearable device 200 includes a measuring unit 210, a GPS receiver 220, a detection information collecting unit 230, a communication unit 240, a non-wearable device access unit 250, and the like.

The measuring unit 210 may include a biosensor measuring biometric information such as body temperature or pulse of a pet while being brought into contact with a body portion of the pet, an acceleration sensor and a gyro sensor that measure movement and direction of the corresponding pet, a camera sensor capturing images in a facing direction of the pet, etc. In addition, the measuring unit 210 may include a temperature detecting sensor measuring temperature of the periphery of the pet, a sound detecting sensor provided to detect a sound signal, etc.

The GPS receiver 220 serves to check a present location of the wearable device 200.

The detection information collecting unit 230 checks the detection information including the biometric information, the movement information, the environmental information, or the combination thereof by referring to data measured by the measuring unit 210 and location information checked by the GPS receiver 220.

Furthermore, when the non-wearable device 700 provides the detection information including the biometric information, movement information, environmental information, etc. according to usage of the non-wearable device 700 of the pet via the non-wearable device access unit 250, the detection information collecting unit 230 may manage the detection information provided from the non-wearable device 700 as the detection information about the behavior and condition of the pet along with the detection information checked based on the data measured by the measuring unit 210 and the GPS receiver 220.

The communication unit 240 performs communication access to the pet monitoring server 100 via the repeater 300, and transmits the detection information checked by the detection information collecting unit 230 to the pet monitoring server 100.

The non-wearable device access unit 250 performs communication access to various non-wearable devices 700 including toys located or installed at a predetermined space, a ride, food container, dishes, a health measurement device, a medical device, etc. via near-field communication (NFC), Bluetooth, BLE, etc.

Furthermore, the non-wearable device access unit 250 receives the detection information about biometric information, movement information, environmental information, etc. detected by the non-wearable device 700 in response to usage of the non-wearable device 700 of the corresponding pet, and provides the received detection information to the detection information collecting unit 230.

FIG. 4 is a view showing a process of transmitting, by the wearable device according to the embodiment of the present disclosure, information detected by a non-wearable device and information directly directed from a pet to a pet monitoring server.

As shown in FIG. 4, a plurality of pets wearing the wearable device 200 around a body portion of the pet may exist in the house, or a plurality of pets are simultaneously located in a predetermined space such as animal hospitals or dog cafes and various non-wearable devices 700 such as toys, medical devices, etc. used by each pet in common may exist in the corresponding space.

In the above case, the pet monitoring server 100 collects and registers the information according to the usage of the non-wearable device of the pet, and must be able to provide the registered information according to a request of the user.

For example, as shown in FIG. 4, it is assumed that a pet 2 and a pet 5 monopolize and play with a pet toy 1 and a pet toy 2, i.e., the non-wearable device 700, and a pet 3 uses a pet medical device, 201.

Then, information about a moving distance of the pet 2 is detected from the non-wearable device 700 installed in the pet toy 1, and the detected information is transmitted to the wearable device 200 worn around the pet 2 via near-field communication (NFC), Bluetooth, BLE, etc., 202.

Next, the wearable device 200 worn around the pet 2 combines the detection information transmitted from the non-wearable device 700 installed at the pet toy 1 with the detection information detected by the wearable device 200 worn around the pet 2, and transmits the combined detection information to the pet monitoring server 100 via the repeater 300, 203. At this point, the detection information generated by the non-wearable device 700 is classified into the detection information of the pet by the wearable device 200 of the corresponding pet and is stored.

In other words, when any one pet among the plurality of pets located in the same space uses the specific non-wearable device 700, the wearable device 200 worn around the specific pet receives the detection information (e.g., moving distance for toy, captured images or test data for medical devices, etc.) according to usage of the pet from the non-wearable device 700. Therefore, the detection information is registered to the pet monitoring server 100 along with the detection information measured separately for each pet and checked.

A process of collection and transmission of the detection information according to the usage of the non-wearable device is applied to a pet 3 and a pet 5 in the same manner as described above.

Accordingly, the user can perform extensive and diverse monitoring on the basis of not only the detection information directly measured from a pet, but also the detection information according to the usage of the non-wearable device 700 of the corresponding pet. In other words, the monitoring region can broadly expand within a wide range including toys of a pet, a variety of equipment of sound, video, sensors, etc. in the house.

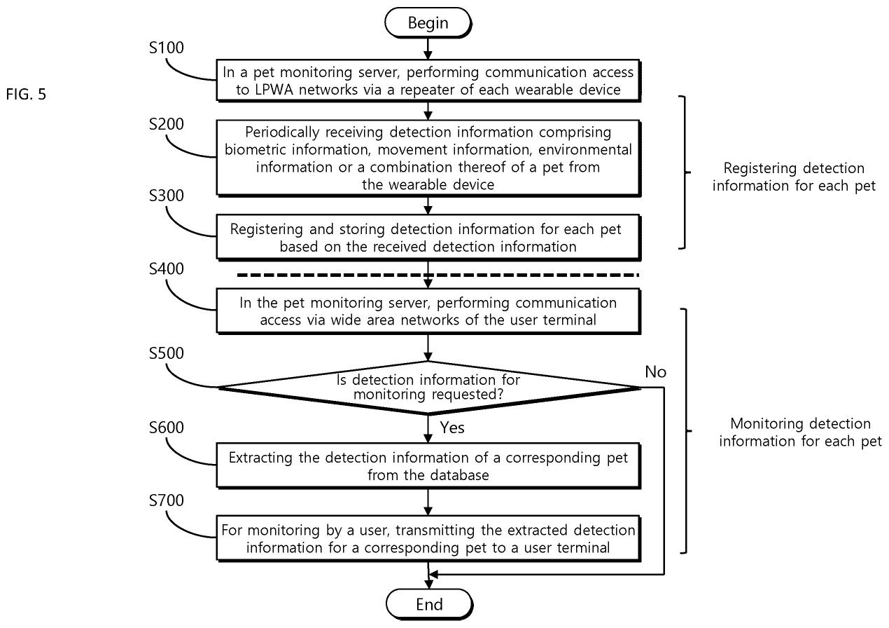

Next, a pet monitoring method according to an embodiment of the present disclosure having the configuration as described above will be describe in detail with reference to FIG. 5. Here, the order of each stage according to the method of the present disclosure may be changed depending on the use environment or a person skilled in the art.

FIG. 5 is a flowchart showing an operation process of the pet monitoring method according to the embodiment of the present disclosure.

As shown in FIG. 5, the pet monitoring server 100 performs communication access via the wearable device 200 worn around a body portion of the pet and the repeater 300, which includes a base station or an access point.

At this point, the pet monitoring server 100 and the wearable device 200 communication-access via the LPWA networks including LoRa, LTE cat, Wi-Fi, NB-IoT.

After the communication access between the pet monitoring server 100 and the wearable device 200 is performed at S100, the pet monitoring server 100 receives the detection information detected by the wearable device 200 of a specific pet, the detection information including biometric information, movement information, environmental information, or combination thereof of the pet, at S200, and classifies the received detection information by each pet and stores and register the detection information in the database 600, at S300.

At this point, when the pet monitoring server 100 receives the detection information, with a non-subscribed user terminal not subscribed in a paid communication service as a repeater, the pet monitoring server 100 may receive the detection information from the wearable device 200 only via Wi-Fi. In addition, with a subscribed user terminal subscribed in a paid communication service as a repeater, the pet monitoring server 100 may receive the detection information from the wearable device 200 via 5G, Wi-Fi, wired or wireless Ethernet, CDMA, LTE, etc., which are the same as described above.

Furthermore, the detection information may include detection information detected directly from a pet by the wearable device 200 worn around the corresponding pet and the detection information detected by and transmitted from the non-wearable device (e.g., houses, dishes, toys, health measurement devices, medical devices, etc. of pet) via near-field communication (NFC), Bluetooth, BLE, etc. The pet monitoring server 100 may receive, with respect to a plurality of pets located in the same space, the corresponding detection information from the wearable device 200 worn around each pet, and the detection information transmitted from the non-wearable device 700 to the wearable device 200 may be classified into detection information of a specific pet and be stored.

Next, while the pet monitoring server 100 periodically receives detection information from the wearable device 200 worn around each pet and the information is stored and managed in the database 600 through the stages from S100 to S300, the pet monitoring server 100 performs communication access via wide area networks of the user terminal 400, at S400, and determines whether or not the user terminal 400 requests detection information for monitoring of a specific pet, at S500.

As a result of determination in S500, when the user terminal 400 requests the detection information for monitoring of the specific pet, the pet monitoring server 100 extracts the detection information of the specific pet stored and managed in the database 600 in S300, at S600), and the extracted detection information of the specific pet is transmitted to the user terminal 400 to perform monitoring related to the behavior or condition of the pet, at S700.

As described above, according to the present disclosure, a signal detected by the wearable device worn around a body portion of a pet is registered to the pet monitoring server via the repeater, and the user accesses the pet monitoring server with the user terminal to remotely monitor the behavior or condition of the pet. Therefore, the wearable device worn the pet and the user terminal are not interdependent, there is no influence on communication traffic, and communication cost can be reduced, and restrictions on time and place are minimized, so that the behavior or condition of the pet can be freely monitored as much as possible.

Furthermore, according to the present disclosure, when a pet uses the non-wearable device capable of being used in common, the wearable device worn around the pet receives information from the non-wearable device and registers the information along with information detected by itself, so that the user accesses the pet monitoring server to perform extensive and diverse monitoring of the behavior or condition of the pet.

As described above, the present disclosure has been described with reference to the embodiment shown in the accompanying drawings, but described for illustrative purposes, and those skilled in the art will understand that various modifications, additions and substitutions are possible, without departing from the scope and spirit of the present disclosure as disclosed in the accompanying claims. Therefore, the patent right of the present disclosure should be defined by the scope and spirit of the invention as disclosed in the accompanying claims.

What is claimed is:

1. A system for monitoring pets comprising:
a pet monitoring server, wherein the pet monitoring server includes:
a first communication access part configured to perform communication access to a wearable device worn around a pet;
a second communication access part configured to perform communication access to a user terminal,
wherein the pet monitoring server is configured to: receive detection information including biometric information, movement information, environmental information, or combination thereof of the pet, detected by the wearable device;
classify and register the received detection information by each pet; and
provide the registered detection information of the pet according to a request of the user terminal, and
wherein the first communication access part and the second communication access part perform communication access through different communication sessions.

2. The system of claim 1, wherein the wearable device is configured to perform communication access to the first communication access part via a repeater including a base station or an access point, and
wherein the first communication access part includes a low power wide area network including at least one of LoRa (long range), LTE cat (long term evolution category), Wi-Fi, or NB-IOT (narrow band internet of things).

3. The system of claim 1, wherein the user terminal is configured to perform communication access to the second communication access part in a web-based or push/pull manner, and
wherein the second communication assess unit is configured to include wide area wired or wireless network including at least one of 5G, Wi-Fi, wired or wireless Ethernet, CDMA, or LTE.

4. The system of claim 1, wherein the pet monitoring server is further configured to receive the detection information from the wearable device via a non-subscribed user terminal not subscribed in a paid communication service as a repeater, and
wherein the non-subscribed user terminal is configured to receive the detection information from the wearable device via near-field communication (NFC), BLE, or Bluetooth, and perform communication access only via Wi-Fi in a process of transmitting the information to the first communication access part.

5. The system of claim 1, wherein the pet monitoring server is further configured to receive the detection information from the wearable device via a subscribed user terminal subscribed in the paid communication service as a repeater, and
wherein the subscribed user terminal is configured to receive the detection information from the wearable device in background environment via near-field communication (NFC), BLE, or Bluetooth, and perform communication access via 5G, Wi-Fi, wired and wireless Ethernet, CDMA, or LTE, in a process of transmitting the information to the first communication access part.

6. The system of claim 1, wherein the detection information includes biometric information, movement information, or environmental information, which is detected by the non-wearable device and is transmitted from the non-wearable device to the wearable device via near-field communication (NFC), BLE, or Bluetooth, and
wherein the non-wearable device includes: a pet house, dishes, toys, health measuring devices, or medical devices.

7. The system of claim 1, wherein the pet monitoring server is further configured to receive the detection information from the wearable device of each pet with respect to a plurality of pets located in the same space, and
wherein the detection information transmitted from the non-wearable device to the wearable device of a specific pet is configured to be classified into the detection information of the specific pet by the specific wearable device and be stored.

8. A method for monitoring pets, the method comprising:
receiving, in a system for monitoring pets, detection information including biometric information, movement information, environmental information, or combination thereof of a pet, wherein the detection information is detected by a wearable device via a first communication access part;
registering the received detection information by each pet; and
transmitting the registered detection information according to a request of a user terminal performing communication access via a second communication access part,
wherein the first communication access part and the second communication access part are configured to perform communication access through different communication sessions.

9. The method of claim 8, wherein the method further comprises:
performing communication access with the first communication access part via a repeater including a base station or an access point; and
wherein the first communication access part includes a low power wide area network including at least one of LoRa (long range), LTE cat (long term evolution category), Wi-Fi, and NB-IOT (narrow band internet of things).

10. The method of claim 8, wherein the user terminal is configured to perform communication access to the second communication access part in a web-based or push/pull manner, and
wherein the second communication assess unit is configured to include wide area wired or wireless network including at least one of 5G, Wi-Fi, wired or wireless Ethernet, CDMA, and LTE.

11. The method of claim 8, wherein the method further comprises:
receiving, in the system for monitoring pets, the detection information from the wearable device via a non-subscribed user terminal not subscribed in a paid communication service as a repeater,
wherein the non-subscribed user terminal is configured to receive the detection information from the wearable device via near-field communication (NFC), BLE, or Bluetooth, and
wherein the non-subscribed user terminal is configured to perform communication access only via Wi-Fi, in a process of transmitting the information to the first communication access part.

12. The method of claim 8, wherein the method further comprises:
receiving, in the system for monitoring pets, the detection information from the wearable device via a subscribed user terminal subscribed in a paid communication service as a repeater, and
wherein the subscribed user terminal is configured to perform communication access via Wi-Fi, wired and wireless Ethernet, CDMA, or LTE, in a process of receiving the detection information in background environment from the wearable device via near-field communication (NFC), BLE, or Bluetooth, and transmitting the information to the first communication access part.

13. The method of claim 8, wherein the detection information further includes biometric information, movement information, or environmental information, which is detected by the non-wearable device and is transmitted from the non-wearable device to the wearable device via near-field communication (NFC), BLE, or Bluetooth, and
wherein the non-wearable device includes: a pet house, dishes, toys, health measuring devices, or medical devices.

14. The method of claim 8, wherein the method further comprises: separately receiving, in the system for monitoring pets, the detection information from the wearable device of each pet, with respect to a plurality of pets located in the same space, and
wherein the detection information transmitted from the non-wearable device to the wearable device of the pet is configured to be classified into the detection information of the pet by the wearable device and be stored.
